(19) United States
(12) Patent Application Publication
(10) Pub. No.: US 2026/0258034 A1
(43) Pub. Date: Sep. 3, 2026

(54) PROCESS FOR THE PREPARATION OF TUCATINIB

(71) Applicant: DR REDDYS LABORATORIES, Telangana (IN)

(72) Inventors: Rajeev Rehani BUDHDEV, Hyderabad (IN); Sekhar NARIYAM MUNASWAMY, Hyderabad (IN); Pradip Kumar SAMANTA, Hyderabad (IN); Sridhar VASAM, Warangal dist (IN); Subbarao CHIKKAM, Konaseema (district) (IN)

(21) Appl. No.: 18/878,808

(22) PCT Filed: Mar. 31, 2023
(86) PCT No.: PCT/IN2023/050319

§ 371 Date: Dec. 24, 2024

(30) Foreign Application Priority Data
Jun. 28, 2022 (IN) 202241037014
Aug. 30, 2022 (IN) 202241049619

(51) Int. Cl. C07D 471/04; B01J 23/44

(52) U.S. Cl. CPC C07D 471/04; B01J 23/44; B01J 2231/641; B01J 2523/824; B01J 2531/90

(57) ABSTRACT

The present application relates to process for the preparation of Tucatinib. The present application also relates to the process for the preparation of Tucatinib intermediates. The present application also provides a process for the preparation of amorphous tucatinib. The present application also provides tucatinib triflate salt characterized by PXRD peaks.

### CROSS REFERENCE

This application claims priority of Indian patent applications IN 202241037014 filed on 28 Jun. 2022 and IN 202241049619 filed on 30 Aug. 2022.

### FIELD OF THE INVENTION

The present application relates to an improved process for the preparation of Tucatinib and intermediates thereof. The present application also relates to the process for the preparation of amorphous Tucatinib.

### BACKGROUND OF THE INVENTION

Tucatinib is the adopted name for a drug chemically described as (N4-(4-([1,2,4]triazolo[1,5-a]pyridin-7-yloxy)-3-methylphenyl)-N6-(4,4-dimethyl-4,5-dihydrooxazol-2-yl)-quinazoline-4,6-diamine and is represented by structural Formula I.

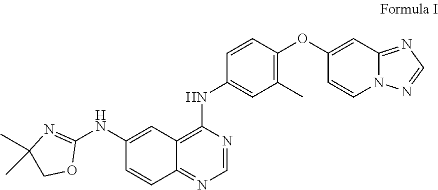 [SMILES: Cc1cc(Nc2ncnc3ccc(NC4=NC(C)(C)CO4)cc23)ccc1Oc1ccn2ncnc2c1]

Tucatinib is a kinase inhibitor and is marketed in USA under the brand name as TUKYSA tablets in 50 mg and 150 mg strengths for the treatment of combination with trastuzumab and capecitabine for treatment of adult patients with advanced unresectable or metastatic HER2-positive breast cancer, including patients with brain metastases, who have received one or more prior anti-HER2-based regimens in the metastatic setting.

U.S. Pat. No. 7,452,895 discloses general procedure for the preparation of Tucatinib and/or its analogues.

U.S. Pat. No. 8,648,087 discloses general procedure for the preparation of Tucatinib and its intermediates.

CN109942576B, CN111825604A, CN112159404A, CN114262327A and CN114230568A describe different processes for the preparation of Tucatinib and its intermediates.

The prior art process for the preparation of Tucatinib have major drawbacks such as difficulties with respect to removal of process related impurities; poor commercial viability due to use of hazardous reactants; use of column chromatography and/or low yields and purity of intermediates and final product. Therefore, there remains a need to develop such a process, which overcomes one or more of the above drawbacks associated with prior art processes for preparation of Tucatinib.

Although methods and materials similar or equivalent to those described herein can be used in the practice or testing of the present application, suitable methods, and materials are described below. All publications, patent applications, patents, and other references mentioned herein are incorporated by reference in their entirety. In addition, the materials, methods, and examples are illustrative only and not intended to be limiting.

### SUMMARY OF THE INVENTION

In first embodiment, the present application provides a one-pot process for the preparation of Tucatinib intermediate of Formula (VI) comprising:

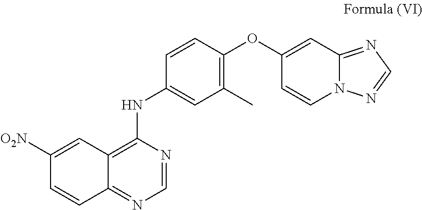 [SMILES: Cc1cc(Nc2ncnc3ccc([N+](=O)[O-])cc23)ccc1Oc1ccn2ncnc2c1]

(a) reacting a compound of Formula (III) with a suitable halogenating reagent to give a compound of Formula (IV);

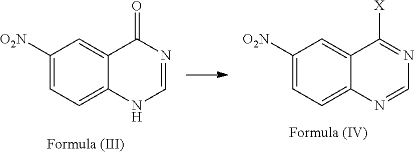

Wherein X is Cl, Br, and I;
(b) coupling a compound of Formula (IV) with a compound of Formula (V) in acidic medium to produce a compound of Formula (VI).

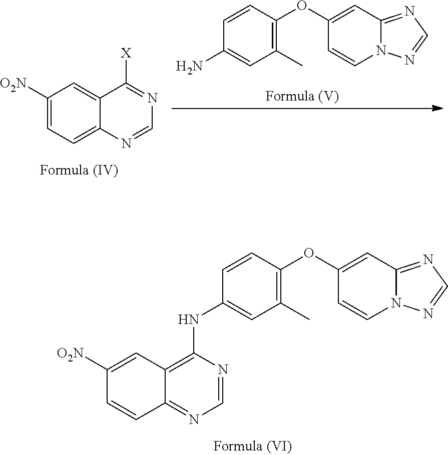

Wherein X is defined above.

In second embodiment, the present application provides a process for the preparation of tucatinib intermediate of Formula (VII) comprising transfer hydrogenation of compound of Formula (VI) to provide a compound of Formula (VII).

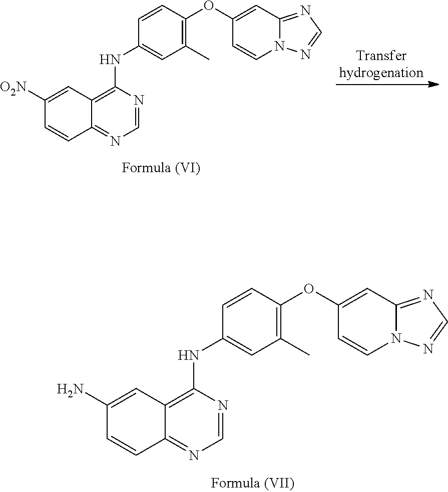 [SMILES: Cc1cc(Nc2ncnc3ccc([N+](=O)[O-])cc23)ccc1Oc1ccn2ncnc2c1 ; Cc1cc(Nc2ncnc3ccc(N)cc23)ccc1Oc1ccn2ncnc2c1]

In third embodiment, the present application provides a process for the preparation of amorphous Tucatinib (I), comprising
a) coupling a compound of Formula (VII) with compound of Formula (VIII) or an acid-addition salt thereof in presence of an organic base to produce Tucatinib Formula (I) or an acid-addition salt thereof; and

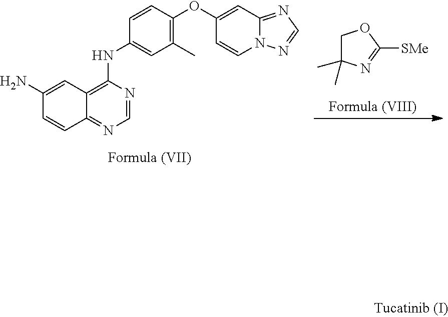

b) optionally, treating the acid-addition salt of compound of formula (I) with a base in a suitable solvent.

In fourth embodiment, the present application provides a novel crystalline form of Tucatinib triflate of formula (IX), which is characterized by:

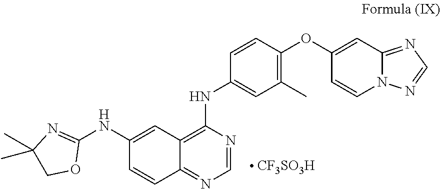

(i) Powder X-ray diffactogram having peaks at about 6.9, 8.5, 10.6, 13.9, 18.4, 21.3, 25.4±0.2±0.2 °2θ;
(ii) DSC thermogram as shown in Figure-2.

### BRIEF DESCRIPTION OF THE DRAWINGS

FIG. 1 is an illustration of a PXRD pattern of crystalline form Tucatinib triflate of formula (IX) obtained as per Example-15.

FIG. 2 is an illustration of DSC thermogram of crystalline form Tucatinib triflate of formula (IX) obtained as per Example-15.

FIG. 3 is an illustration of PXRD pattern of amorphous form of Tucatinib of formula (I) as per Example-20.

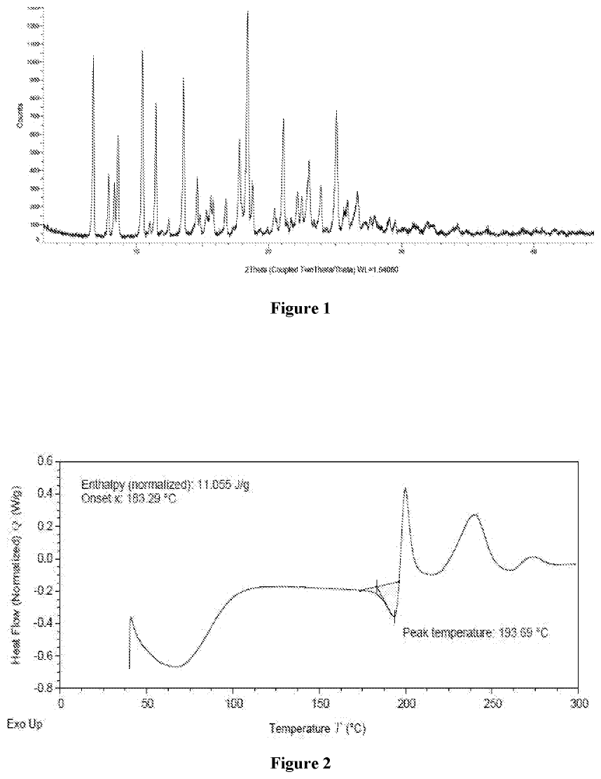

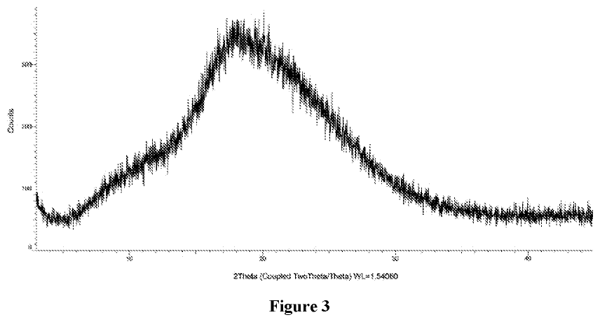

### DETAILED DESCRIPTION OF THE INVENTION

As used herein, “comprising” means the elements recited, or their equivalent in structure or function, plus any other element or elements which are not recited. The terms “having” and “including” are also to be construed as open ended unless the context suggests otherwise.

All ranges recited herein include the endpoints, including those that recite a range “between” two values.

Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

The starting materials used in this aspect, 6-nitro-4(3H)-quinazolinone (herein referred as compound of Formula (III)), may be obtained according to any method known in the art or may be procured from the commercially available sources.

In first embodiment, the present application provides a one-pot process for the preparation of Tucatinib intermediate of Formula (VI) comprising:

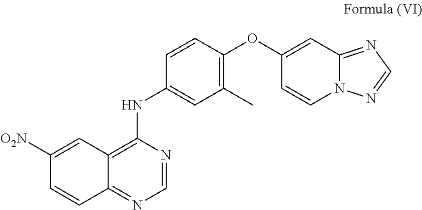 [SMILES: Cc1cc(Nc2ncnc3ccc([N+](=O)[O-])cc23)ccc1Oc1ccn2ncnc2c1]

(a) reacting a compound of Formula (III) with a suitable halogenating reagent to give a compound of Formula (IV);

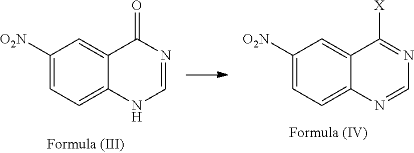

Wherein X is Cl, Br, and I;
(b) coupling a compound of Formula (IV) with a compound of Formula (V) in acidic medium to produce a compound of Formula (VI).

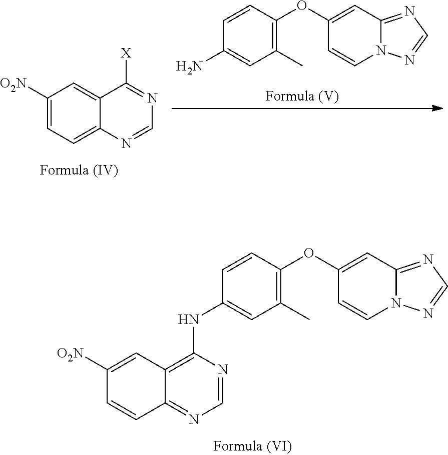

Wherein X is defined above.

Suitable halogenating reagent used in step a) may include but not limited to phosphorous oxychloride, thionyl chloride, phosphorous pentachloride, phosphorus trichloride, phosphorus oxybromide, phosphorus tribromide, bromine, iodine, iodine chloride and the like. In one specific embodiment, the suitable halogenating reagent may be a suitable chlorinating agent. Suitable chlorinating agent used in step a) include, but not limited to phosphorous oxychloride, chlorine, phosphorous pentachloride, thionyl chloride or any other halogenating agents. Specifically, the suitable chlorinating agent may be phosphorous oxychloride.

The above step a) reaction may be carried out in the presence of suitable organic solvent and base.

Suitable base used in step a) include, but are not limited to pyridine, piperidine, pyrimidine, triethylamine, tributylamine, N-methylmorpholine, N,N-diisopropylethylamine, diethylamine, 2,2-bipyridine, 1,1,3,3-tetramethylguanidine, DBU, DABCO or the like. Specifically, suitable base may be N,N-diisopropylethylamine. Alternatively, the suitable base may be triethylamine.

Suitable organic solvent used in step a) include, but are not limited to hydrocarbon solvents, such as toluene, xylene, chlorobenzene, tetralin and the like; alcoholic solvents such as methanol, ethanol, isopropyl alcohol, n-butanol, 1-propanol and the like; ketone solvents, such as acetone, ethyl methyl ketone, diethyl ketone, methyl isobutyl ketone, C₃-C₆ ketones and the like; halogenated hydrocarbons such as dichloromethane, chloroform and the like; aliphatic hydrocarbon solvents, such as n-pentane, n-hexane, n-heptane and the like; ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1,4-dioxane and the like; nitrile solvent, such as acetonitrile, propionitrile, C₂-C₆ nitriles and the like; ester solvents, such as ethyl formate, methyl acetate, ethyl acetate, propyl acetate, butyl acetate, methyl propanoate, ethyl propanoate, methyl butanoate, ethyl butanoate, and the like; polar aprotic solvents such as dimethyl formamide, dimethylacetamide, N-methylpyrrolidine (NMP), formamide, acetamide, propanamide, dimethyl sulfoxide and the like; water and mixtures thereof.

The intermediate of step a), compound of formula (IV) may be directly used for the next step without isolation for the next step.

The above step b) reaction may be carried out in the presence of suitable organic solvent. Preferably, the solvent used in step a) may be used in step b). The pH of the reaction medium in step b) may be about 1-6. Specifically, the pH of the reaction medium in step b) is 3-5. An acid may be added to the reaction mass to maintain the pH of the reaction mass of step b). Specifically, the acid may be hydrochloric acid.

The intermediate, compound of Formula (VI) may be isolated from the reaction mass by a method known in the art. Specifically, compound of Formula (VI) may be isolated by quenching the reaction mass with an anti-solvent like water and filtering the required product. Optionally, the compound of formula (VI) may be purified by crystallization from a solvent or a mixture of solvents.

The compound of Formula (VI) may be converted to tucatinib by a method known in the art.

The prior art (CN114230568A) process describes the coupling of a compound of Formula (IV) with Formula (V) in basic medium (in presence of potassium carbonate) to give a compound of Formula (VI) with yield of 88%-90%. The inventors of the present application found that the coupling of a compound of Formula (IV) with Formula (V) in acidic medium (preferably hydrochloric acid) to give a compound of Formula (VI) with more yield when compared to the prior art.

In second embodiment, the present application provides a process for the preparation of tucatinib intermediate of Formula (VII) comprising transfer hydrogenation of compound of Formula (VI) to provide a compound of Formula (VII).

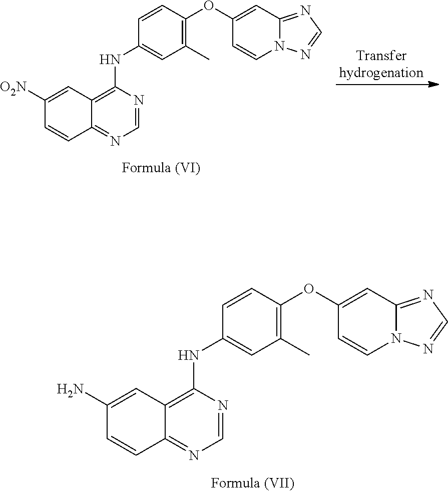 [SMILES: Cc1cc(Nc2ncnc3ccc([N+](=O)[O-])cc23)ccc1Oc1ccn2ncnc2c1 ; Cc1cc(Nc2ncnc3ccc(N)cc23)ccc1Oc1ccn2ncnc2c1]

The above transfer hydrogenation reaction may be carried out in the presence of suitable organic solvent and reagent.

Transfer hydrogenation may be carried out in presence of metal catalyst including but not limited to palladium, raney nickel, iron, platinum and the like.

Suitable reagent used in transfer hydrogenation includes but not limited to ammonium formate, triethyl silane, isopropyl alcohol, formic acid, hydrazine and the like.

Suitable organic solvent used in transfer hydrogenation step include, but are not limited to polar aprotic solvents such as dimethyl formamide, dimethylacetamide, N-methylpyrrolidine (NMP), formamide, acetamide, propanamide, dimethyl sulfoxide and the like; ketone solvents, such as acetone, ethyl methyl ketone, diethyl ketone, methyl isobutyl ketone, C₃-C₆ ketones and the like; hydrocarbon solvents, such as toluene, xylene, chlorobenzene, tetralin and the like; alcoholic solvents such as methanol, ethanol, isopropyl alcohol, n-butanol, 1-propanol and the like; halogenated hydrocarbons such as dichloromethane, chloroform and the like; aliphatic hydrocarbon solvents, such as n-pentane, n-hexane, n-heptane and the like; ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1,4-dioxane and the like; nitrile solvent, such as acetonitrile, propionitrile, C₂-C₆ nitriles and the like; ester solvents, such as ethyl formate, methyl acetate, ethyl acetate, propyl acetate, butyl acetate, methyl propanoate, ethyl propanoate, methyl butanoate, ethyl butanoate, and the like; water and mixtures thereof.

The prior art process for this reduction step requires hydrogenation at high pressure reactor and in presence of hydrogen gas. This may cause some safety issues in the manufacturing plant due to use of flammable material like hydrogen and closed reactor like high pressure reactor. The inventors of the present application found that reduction by transfer hydrogenation using a metal catalyst and suitable reagent, which generates hydride, makes the process safer and simpler.

The compound of Formula (VI) may be converted to tucatinib by a method known in the art.

The compound of Formula (VII) may be converted to tucatinib by a method known in the art.

In third embodiment, the present application provides a process for the preparation of amorphous Tucatinib (I), comprising
a) coupling a compound of Formula (VII) with compound of Formula (VIII) or an acid-addition salt thereof in presence of an organic base to produce Tucatinib Formula (I) or an acid-addition salt thereof; and

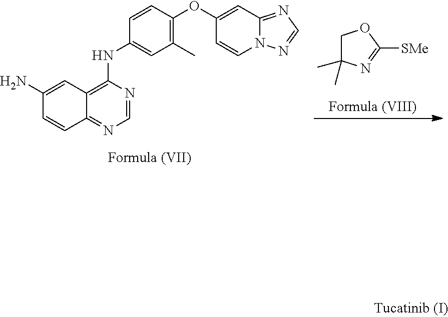

b) optionally, treating the acid-addition salt of compound of formula (I) with a base in a suitable solvent.

The acid-addition salt of compound of formula (VIII) may be a hydrochloride salt, oxalic acid salt, triflic acid salt, sulfuric acid salt, maleic acid salt, malonic acid salt and the like. Specifically, the acid-addition salt of compound of formula (VIII) may be a hydrochloride salt and triflic acid salt.

The organic base which may be used in step a) may be selected from a group of basic solvents like pyridine, 2-picoline, 3-picoline, 4-picoline, pyrrole, pyrrolidine, piperidine, imidazole, triethylamine, diisopropyl ethyl amine, 1,4-diazabicyclo[2.2.2]octane, 1,8-diaza-bicyclo(5.4.0)undec-7-ene, pyrimidine and mixture thereof, optionally along with a suitable organic solvent.

Suitable organic solvent used in step a) may be selected from a group of halogenated hydrocarbons such as dichloromethane, chloroform and the like; ester solvents, such as ethyl formate, methyl acetate, ethyl acetate, propyl acetate, butyl acetate, methyl propanoate, ethyl propanoate, methyl butanoate, ethyl butanoate, and the like; hydrocarbon solvents, such as toluene, xylene, chlorobenzene, tetralin, hexane, heptane and the like; and mixtures thereof.

Step-a) reaction may be performed at any temperature. Specifically, step a) may be performed at about 30° C. to about 80° C.

The acid-addition salt of Tucatinib (I) may be the same acid-addition salt, as in compound of Formula (VIII). Specifically, acid-addition salt of Tucatinib (I) may be a hydrochloride salt and triflic acid salt.

The pKa of Tucatinib is 6.61, the pKa of pyridine is 5.25, and the pKa of compound of Formula (VIII) is 5.36. Hence, it is expected that the acid-addition salt of compound of Formula (VIII) is transferred to Tucatinib during the course of step a) reaction. Hence, an acid-addition salt of compound of Formula (VIII) results into the same acid-addition salt of Tucatinib. However, if the reaction of compound of Formula (VII) and acid-addition salt of compound of Formula (VIII) is quenched with water, then Tucatinib of formula (I) is isolated.

The acid-addition salt of tucatinib as prepared in step a) may optionally be crystallized before step b) from suitable solvent or a mixture thereof.

In step b), the acid-addition salt of compound of Formula (I) may be treated with a base in presence of a suitable solvent to provide amorphous tucatinib.

Suitable base used in the above embodiment include, but are not limited to sodium carbonate, potassium carbonate, sodium bicarbonate, potassium bicarbonate, sodium hydroxide, potassium hydroxide, lithium hydroxide, ammonia, organic bases such as triethylamine, pyridine and the like. Specifically, the base may be ammonia.

The solvent may be selected from a group of polar aprotic solvent such as dimethyl formamide, dimethylacetamide, N-methylpyrrolidine (NMP), formamide, acetamide, propanamide, dimethyl sulfoxide and the like. Specifically, the solvent is dimethyl formamide.

In fourth embodiment, the present application provides a novel crystalline form of Tucatinib triflate of formula (IX), which is characterized by:

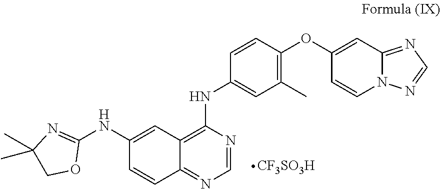

(i) Powder X-ray diffactogram having peaks at about 6.9, 8.5, 10.6, 13.9, 18.4, 21.3, 25.4±0.2 °2θ;
(ii) DSC thermogram as shown in Figure-2.

In one embodiment, the present application provides use of Tucatinib triflate of formula (IX) characterized by PXRD peaks at about 6.9, 8.5, 10.6, 13.9, 18.4, 21.3, 25.4±0.2 °2θ for the preparation of amorphous form of tucatinib (I).

The removal of solvent at any stage of the process of the present application may be carried out by methods known in the art or any procedure disclosed in the present application. In preferred embodiments, removal of solvent may include, but not limited to: solvent evaporation or sublimation under atmospheric pressure or reduced pressure/vacuum such as a rotational distillation using Büchi® Rotavapor®, spray drying, freeze drying (Lyophilization), agitated thin film drying and the like.

The compounds at any stage of the process of the present application may be isolated using conventional techniques known in the art. For example, useful techniques include but are not limited to, decantation, centrifugation, gravity filtration, suction filtration, concentrating, cooling, stirring, shaking, combining with an anti-solvent, adding seed crystals, evaporation, flash evaporation, simple evaporation, rotational drying, spray drying, thin-film drying, freeze-drying, or the like. The isolation may be optionally carried out at atmospheric pressure or under reduced pressure. The solid that is obtained may carry a small proportion of occluded mother liquor containing a higher percentage of impurities and, if desired, the solid may be washed with a solvent to wash out the mother liquor.

The compounds at any stage of the process of the present application may be recovered from a suspension/solution using any of techniques such as decantation, filtration by gravity or by suction, centrifugation, slow evaporation, or the like, or any other suitable techniques. The reaction can be efficiently completed at room temperature or ambient temperature or if required reaction mass can be heated to elevated temperatures or up to about the reflux temperatures, and maintained for about 10 minutes to about 5 hours or longer.

The resulting solid may be optionally further dried. Drying may be suitably carried out using equipment such as a tray dryer, vacuum oven, air oven, fluidized bed dryer, spin flash dryer, flash dryer, or the like, at atmospheric pressure or under reduced pressure. Drying may be carried out at temperatures less than about 100° C., less than about 60° C., less than about 40° C., or any other suitable temperatures, at atmospheric pressure or under reduced pressure, and in the presence or absence of an inert atmosphere such as nitrogen, argon, neon, or helium. The drying may be carried out for any desired time periods to achieve a desired purity of the product, such as, for example, about 1 to about 15 hours, or longer.

Certain specific aspects and embodiments of the present application will be explained in greater detail with reference to the following examples, which are provided only for purposes of illustration and should not be construed as limiting the scope of the application in any manner.

### EXAMPLES

### Example 1: Preparation of 6-Nitro-4(3H)-quinazolinone (III)

2-Amino-5-nitrobenzoic acid (6 g) in formamide (14.84 g) were charged into a reactor at 29° C. under nitrogen atmosphere. Acetic acid (9.89 g) was added to the reaction mass at 29° C. The reaction mass was heated to 120° C. and the reaction mass was stirred for 16 hours at 110-120° C. After completion of the reaction the reaction mass was cooled to 20-30° C. Purified water (60 mL) was added to the reaction mass at 29° C. and stirred for 60 minutes. The reaction mass was filtered and washed with purified water (18 mL) and suck dried for 20 minutes. The obtained material was dried at 50-55° C. under vacuum to obtain the title compound as yellow solid.

Yield: 79.4%

### Example-2: Preparation of 4-Chloro-6-nitroquinazoline (IV)

6-nitroquinazolin-4(1H)-one (III) and chlorobenzene (20 mL) were charged into a reactor at 28° C. under nitrogen atmosphere. N-ethyl-N-isopropylpropan-2-amine (DIPEA; 1.488 g) were charged into the reaction mixture at 28° C. and stirred for 5 minutes. Phosphorus oxychloride (1.203 g) was slowly added to the reaction mass at 29° C. and stirred for 5 minutes. The reaction mass was heated to 95° C. and maintained for 3-4 hours. The reaction mass was cooled to 28° C. and stirred for 10 minutes. 10% sodium bicarbonate solution (20 mL) was added to the reaction mass and stirred for 10 minutes. Both layers were separated. Filtered the unwanted material from reaction mass. Both layers were separated. Aqueous layer was extracted with chlorobenzene. Total organic layer was washed with water (10 mL). Both layers were separated and the organic layer was proceeded for next step.

### Example-3: Preparation of N-(4-([1,2,4]triazolo[4,3-a]pyridin-7-yloxy)-3-methylphenyl)-6-nitroquinazolin-4-amine (VI)

4-([1,2,4]triazolo[4,3-a]pyridin-7-yloxy)-3-methylaniline ((V); 0.5 g) and isopropyl alcohol (5 mL) were charged into a reactor at 28° C. 4-chloro-6-nitroquinazoline ((IV); 0.480 g) and N-ethyl-N-isopropylpropan-2-amine (DIPEA; 0.323 g) were charged at 28° C. and stirred for 5-10 minutes. The reaction mass was heated to 81° C. and maintained for 7-8 hours. The solvent from the reaction mass distilled completely at 55° C. The reaction mass was cooled to 29° C. and water (10 mL) was added to the reaction mass and maintained for 2-3 hours. Filtered the solid and washed with water (10 mL) under vacuum and suck dried for 20 minutes. The obtained material was dried at 50-55° C. under vacuum to obtain the title compound.

Yield: 80%

### Example-4: Preparation of N-(4-([1,2,4]triazolo[1,5-a]pyridin-7-yloxy)-3-methylphenyl)-6-nitroquinazolin-4-amine (VI)

4-([1,2,4]triazolo[4,3-a]pyridin-7-yloxy)-3-methylaniline ((V); 5 g) and 1-butanol (150 mL) were charged into a reactor at 28° C. 4-chloro-6-nitroquinazoline ((IV); 5.67 g) was added to the reaction mixture 28° C. and stirred for 5-10 minutes. Con. HCl (0.759 g) was slowly added to the reaction mass at 29° C. The reaction mass was heated to 80-90° C. and stirred at this temperature for 2 hours. After reaction completion, the reaction mass was cooled to 29° C. and stirred for 60 minutes at 29° C. Filtered the obtained solid and washed with 1-butanol (25 mL) and suck dried for 2 hours. The obtained wet material was purified by using 10% aqueous NaOH solution in acetone at 35-45° C. to obtain the title compound.

Yield: 93%

### Example-5: Preparation of N-(4-([1,2,4]triazolo[1,5-a]pyridin-7-yloxy)-3-methylphenyl)-6-nitroquinazolin-4-amine (VI)

6-nitroquinazolin-4(1H)-one (III) and chlorobenzene (20 mL) were charged into a reactor at 28° C. under nitrogen atmosphere. N-ethyl-N-isopropylpropan-2-amine (DIPEA; 1.488 g) were charged into the reaction mixture at 28° C. and stirred for 5 minutes. Phosphoryl trichloride (1.203 g) was slowly added to the reaction mass at 29° C. and stirred for 5 minutes. The reaction mass was heated to 95° C. and maintained for 3-4 hours. The reaction mass was cooled to 28° C. and stirred for 10 minutes. 10% sodium bicarbonate solution (20 mL) was added to the reaction mass and stirred for 10 minutes. Both layers were separated. Filtered the unwanted material from reaction mass. Both layers were separated. Aqueous layer was extracted with chlorobenzene. Total organic layer was washed with water (10 mL). Both layers were separated and the organic layer was proceeded for next step.

4-([1,2,4]triazolo[4,3-a]pyridin-7-yloxy)-3-methylaniline ((V); 1 g) was added to the above organic layer at 29° C. and stirred for 5 minutes. The reaction mass was heated to 80-90° C. and continued to stirred for 8 hours. After completion of the reaction, the reaction mass was cooled to 20-30° C. Acetone (20 mL) was added to the reaction mass and stirred for 60 minutes at 25-30° C. Filtered the obtained solid and washed with acetone (5 mL) under vacuum and suck dried for 20 minutes. The obtained crude material was purified by using methanol (5 mL) to obtain the title compound.

Yield: 72%.

### Example-6: Preparation of N-(4-([1,2,4]triazolo[1,5-a]pyridin-7-yloxy)-3-methylphenyl)-quinazoline-4,6-diamine (VII)

N-(4-([1,2,4]triazolo[1,5-a]pyridin-7-yloxy)-3-methylphenyl)-6-nitroquinazolin-4-amine (VI; 0.5 g) and DMF (10 mL) were charged into a reactor at 28° C. under nitrogen atmosphere. Ammonium formate (0.557 g) was slowly added to the reaction mass at 25-30° C. and stirred for 5 minutes. 10% palladium on carbon (0.064 g) was slowly added to the reaction mass at 25-30° C. and stirred for 5 minutes. The reaction mass was heated to 45-55° C. and maintained for 60 minutes at 45-55° C. After completion of the reaction, the reaction mass was cooled to 20-30° C. The reaction mixture was then filtered through a Celite® pad, the cake was washed with DMF (4 mL). The combined filtrate was poured into purified water (40 mL), filtered and washed it with water (5.0 mL), suck dried for 60 minutes. The obtained material was dried at 50-55° C. under vacuum to obtain the title compound.

Yield: 64.8%.

### Example-7: Preparation of Tucatinib (I)

N-(4-([1,2,4]triazolo[1,5-a]pyridin-7-yloxy)-3-methylphenyl)-quinazoline-4,6-diamine (VII; 0.1 g), 4,4-dimethyl-2-(methylthio)-4,5-dihydrooxazole (VIII; 0.152 g) and DMF (1 mL) were charged in to 28° C. under nitrogen atmosphere and stirred for 5 minutes. Cesium carbonate (0.062 g) was slowly added to the reaction mass at 28° C. and stirred for 5 minutes. The reaction mass was heated to 120-130° C. and maintained for 22 hours at 126° C. After completion of the reaction, reaction mass was cooled to 20-30° C. The reaction mixture was poured into purified water (3 mL). The obtained solid was filtered and washed with purified water (2 mL), suck dried followed by dried under vacuum at 55° C. to give Tucatinib of Formula (I) as yellow solid.

### Example-8: Preparation of 4,4-dimethyloxazolidine-2-thione (X)

2-Amino-2-methylpropan-1-ol (IXa; 10 g) in toluene (20 mL) and Carbon disulfide (9.39 g) were charged in to 28° C. under nitrogen atmosphere and stirred for 5 minutes. The reaction mas was diluted with water (25 mL) and separated the aqueous layer and aqueous layer was taken as such for cyclization reaction.

Above aqueous layer was treated with ~10% aq. Sodium hydroxide solution (2.5 mL) at 20-30° C. The reaction mass was heated to 90-100° C. and maintained for 2 hours at same temperature. The reaction mas was cooled to 0-10° C. and maintained for 2 hours at same temperature. The obtained solid was filtered and washed with chilled water (5 ml) and suck dried for 2 hours. Suck dried material was purified by using n-hexane to obtain title compound as off white solid.

Yield: 3.35 g

### Example-9: Preparation of 4,4-Dimethyl-2-(methylthio)-4,5-dihydrooxazole (VIII)

To a solution of 4,4-dimethyloxazolidine-2-thione ((X); 10 g) in 5% aqueous NaOH solution (250 mL) were charged in to 28° C. under nitrogen atmosphere and stirred for 5 minutes. Dimethylsulfate (30.74 g) was added to the reaction mass at 29° C. and stirred for 2 hours at same temperature. The reaction mass heated to 40° C. and maintained for 4 hours. The reaction mas was diluted with EtOAc (400 mL) and separated the organic layer. The organic layer was distilled to afford the title compound as an oil.

Yield: 5.5 g

### Example-10: Preparation of N-(4-([1,2,4]triazolo[1,5-a]pyridin-7-yloxy)-3-methylphenyl)-6-nitroquinazolin-4-amine (VI)

6-Nitroquinazolin-4(1H)-one (III; 1 g) and 4-([1,2,4]triazolo[4,3-a]pyridin-7-yloxy)-3-methylaniline (V; 1 g) and chlorobenzene (20 mL) were charged into a reactor at 28° C. under nitrogen atmosphere. N-ethyl-N-isopropylpropan-2-amine (DIPEA; 1.488 g) were charged into the reaction mixture at 28° C. and stirred for 5 minutes. Phosphoryl trichloride (1.203 g) was slowly added to the reaction mass at 29° C. and stirred for 5 minutes. The reaction mass was heated to 85° C. and maintained for 8-9 hours. The reaction mass was cooled to 35° C. and stirred for 5 minutes. Acetone (10 mL) was added to the reaction mass at 29° C. and maintained for 3-4 hours. 10% NaOH solution was added to the reaction mass at 29° C. and maintained for 2-3 hours. The obtained solid was filtered and washed with acetone (10 mL), suck dried and dried at 55° C. under vacuum to obtain the title compound.

Yield: 1.1 g

### Example 11: Preparation of Tucatinib (I)

To a solution of 4,5-dihydro-4,4-dimethyl-2-(methylthio)oxazole trifluoromethanesulfonic acid salt (VIIIa; 23.10 g) in pyridine (100 mL) was added to N-(4-([1,2,4]triazolo[1,5-a]pyridin-7-yloxy)-3-methylphenyl)-quinazoline-4,6-diamine (VII; 10 g) at 27° C. and maintained for 48 hours at same temperature. After completion of the reaction, purified water (200 mL) was slowly added to the reaction mass, extracted with MDC (200 mL) and washed with 10% aqueous NaHCO₃ (100 mL), followed by 10% aqueous NaCl (50 mL). Both layers were separated and the organic layer was distilled below at 35° C. to afford crude tucatinib, followed by slurried with water and isolated from ethanol, suck dried followed by dried under vacuum at 50° C. to give the title compound of Formula (I) as a yellow solid.

Yield: 9.5 g

### Example 12: Preparation of Tucatinib (I)

To a solution of 4,5-dihydro-4,4-dimethyl-2-(methylthio)oxazole trifluoromethanesulfonic acid salt (VIIIa; 11.5 g) in picoline (50 mL) was added to N-(4-([1,2,4]triazolo[1,5-a]pyridin-7-yloxy)-3-methylphenyl)-quinazoline-4,6-diamine (VII; 5 g) at 28° C. and maintained for 48 hours at same temperature. After completion of the reaction, purified water (100 mL) was slowly added to the reaction mass, extracted with MDC (100 mL) and washed with 10% aqueous NaHCO₃ (50 mL), followed by 10% aqueous NaCl (25 mL). Both layers were separated and the organic layer was distilled below at 35° C. to afford crude tucatinib, followed by slurried with water and isolated from ethanol, suck dried followed by dried under vacuum at 50° C. to give the title compound of Formula (I) as a yellow solid.

Yield: 4.8 g

### Example-13: Preparation of N-(4-([1,2,4]triazolo[4,3-a]pyridin-7-yloxy)-3-methylphenyl)-6-nitroquinazolin-4-amine (VI)

6-nitroquinazolin-4(1H)-one (III) (11.3 Kg) and toluene (12.0 L) were charged into a reactor at 25-35° C. under nitrogen atmosphere. Triethylamine (13.1 Kg) were charged into the reaction mixture at 25-35° C. and stirred for 10-15 minutes. Phosphorus oxychloride (13.6 Kg) was slowly added to the reaction mass at 25-35° C. and stirred for 10-15 mins. The reaction mass was heated to 90-95° C. and maintained for 3-4 hours. After completion of the reaction, reaction mass was cooled to 25-35° C. In an another reactor sodium bicarbonate (7.5 kg) and DM water (150.0 L) charged at 25-35° C. and stirred for 10-15 mins to get clear solution. The reaction mass slowly added to the above prepared solution at 25-35° C. and stirred for 10-15 mins. Celite® (5.0 kg) charged to the reactor and stirred for 20-30 mins. Filtered and residue was washed with toluene (50.0 L). Filtrate charged to the reactor at 25-35° C. and allowed to settle for 30-40 mins and layers separated. Organic layer was taken as such for next step without further purification.

4-([1,2,4]triazolo[4,3-a]pyridin-7-yloxy)-3-methylaniline ((V); 10.0 Kg) was added to the reactor containing above organic layer at 25-35° C. Toluene (50.0 L) was charged to the reactor and stirred for 10-15 mins. The reaction mass was heated to 80-90° C. and continued to stir for 12 hours. After completion of the reaction, the reaction mass was cooled to 25-35° C. Filtered the reaction mass and washed with toluene (50.0 L) to obtain N-(4-([1,2,4]triazolo[1,5-a]pyridin-7-yloxy)-3-methylphenyl)-6-nitroquinazolin-4-amine hydrochloride.

N-(4-([1,2,4]triazolo[1,5-a]pyridin-7-yloxy)-3-methylphenyl)-6-nitroquinazolin-4-amine hydrochloride suspended into DMF (93.0 L) at 25-35° C. Stirred for 15-20 mins and slowly added ~5% w/w aq. NaHCO₃ solution (279.0 L) into the reactor at 20-30° C. Stirred for 2 hours, filtered and washed with DM water (2×93.0 L). Suck dried for 3 hours and further dried under vacuum at 45-55° C. for 12 hours to obtain the title compound.

Yield: 92.4%.

HPLC purity: 99%

### Example-14: Preparation of N-(4-([1,2,4]triazolo[1,5-a]pyridin-7-yloxy)-3-methylphenyl)-quinazoline-4,6-diamine (VII)

N-(4-([1,2,4]triazolo[1,5-a]pyridin-7-yloxy)-3-methylphenyl)-6-nitroquinazolin-4-amine (VI; 12.0 kg) and DMF (120.0 L) were charged into the reactor at 25-35° C. under nitrogen atmosphere. Ammonium formate (8.23 kg) charged to the reactor contents at 25-35° C. and stirred for 5-10 mins. 10% palladium on carbon (50% wet) (1.2 kg) was slowly added to the reaction mass at 25-35° C. and stirred for 5 to 10 mins. The reaction mass was heated to 65-75° C. and maintained for 4 hours at 65-75° C. After completion of the reaction, the reaction mass was cooled to 25-35° C. and filtered through Celite® pad, the wet cake washed with DMF (24.0 L). The combined filtrate was charged the reactor at 25-35° C. and slowly added purified water (300.0 L) into the reactor. The resulting suspension was filtered and wet cake washed with water (60.0 L), suck dried for 3 hours. The obtained compound was dried at 50-55° C. under vacuum to obtain the title compound as pale green solid.

Yield: 90%.

HPLC purity: 99%

### Example 15: Preparation of Tucatinib Triflate Salt (IX)

4,5-dihydro-4,4-dimethyl-2-(methylthio)oxazole trifluoromethanesulfonic acid salt (VIIIa; 11.38 kg) and pyridine (24.75 L) charged to the reactor at 25-35° C. and stirred for 30-40 mins. Charged N-(4-([1,2,4]triazolo[1,5-a]pyridin-7-yloxy)-3-methylphenyl)-quinazoline-4,6-diamine (4.95 kg) and DCM (49.5 L) into the reactor at 25-35° C. and stirred for 120 hours. After completion of the reaction, the reaction mass filtered and wet cake washed with DCM (24.75 L). Suck dried for 3 hours and further dried for 12 hours at 45-55° C. to obtain Tucatinib triflate salt.

Yield: 95%

HPLC purity: 99%

### Example 16: Preparation of Tucatinib Triflate Salt (IX)

To a solution of N-(4-([1,2,4]triazolo[1,5-a]pyridin-7-yloxy)-3-methylphenyl)-quinazoline-4,6-diamine (VII; 20 g) in Ethyl acetate (200 ml) was added to the solution of 4,5-dihydro-4,4-dimethyl-2-(methylthio)oxazole trifluoromethanesulfonic acid salt (VIIIa; 30.8 g) in 2-picoline (100 mL) at 25-35° C. Heated the reaction mass to 50° C. and stirred for 20 hours. After completion of the reaction, reaction mass filtered and washed with ethyl acetate (100 ml), suck dried followed by dried under vacuum at 40-50° C. to obtain Tucatinib triflate salt.

Yield: 86%

HPLC purity: 99.5%

### Example 17: Purification of Tucatinib Triflate Salt (IX)

Tucatinib triflate salt (10 g) was added into a mixture of DMF (20 mL) and ethyl acetate (60 mL) at 25-35° C. and stirred for 1 hour. The resulting yellow suspension was filtered and washed with ethyl acetate (2×10 mL), suck dried and further dried for 8 hours to obtain Tucatinib triflate salt.

Yield: 86%

HPLC purity: 99.5%

### Example-18: Preparation of Tucatinib (I)

Tucatinib Triflate salt (6.5 kg) and DMF (19.5 L) were charged into the reactor at 25-35° C. and stirred for 30-40 mins to get clear solution. Slowly added ~5% w/w aq. NaHCO₃ solution (78.0 L) into the reactor at 20-30° C. Stirred for 4 hours, filtered and washed with DM water (2×32.5 L). Suck dried for 3 hours and further dried under vacuum at 45-55° C. for 12 hours to obtain the title compound.

Yield: 95%

HPLC purity: 99.8%

### Example 19: Preparation of Amorphous Form of Tucatinib (I)

Tucatinib Triflate salt (5 g) dissolve in DMF (10 ml) in a RBF and the solution was added into aq. Ammonia solution at 20-30° C. and maintained for 1 hour at same temperature. Filtered the reaction mass and washed with water (10 ml), suck dried followed by dried under vacuum at 50° C. to give the title compound.

Yield: 86%

HPLC purity: 99.5%

### Example-20: Preparation of Amorphous Form of Tucatinib (I)

Tucatinib (3 g) suspended in water (30 mL) at 20-30° C. and mass temperature was cooled to 5-10° C. Added slowly, Trifluoroacetic acid (5.9 g) into the suspension at same temperature and maintained for 10 min for dissolution. The above solution was added drop wise into aq. Ammonia solution (60 mL) at same temperature and maintained for 30 min. Filtered the reaction mass and washed it with water (20 mL), suck dried followed by dried under vacuum at 50° C. to give the title compound.

Yield: 90%

### Example-21: Preparation of Amorphous Form of Tucatinib (I)

Tucatinib (2 g) suspended in water (20 mL) at 20-30° C. and mass temperature was cooled to 5-10° C. Added slowly, ~6N HCl (2.5 mL) into the suspension at same temperature and maintained for 10 min for dissolution. The above solution was added drop wise into aq. Ammonia solution (40 mL) at same temperature and maintained for 30 min. Filtered the mass and washed it with water (20 mL), suck dried followed by dried under vacuum at 50° C. to give the Tucatinib Amorphous.

Yield: 90%

Purity: 99.0%

### Example-22: Preparation of 4,5-dihydro-4,4-dimethyl-2-(methylthio)oxazole trifluoromethanesulfonic acid (VIIIa)

4,4-dimethyloxazolidine-2-thione (1.5 KG) was charged in DCM (7.5 L) at 25-35° C., Cooled the mass temperature to 0-5° C. and slowly added methyl trifluoromethanesulfonate (2.15 Kg) at 0-5° C. and maintained for 20 min at same temperature. Allowed the reaction mass temperature to 25-35° C. and maintained for 2 hours at same temperature. Distill the DCM up to 1-2 vol at below 30° C. and charge MTBE (10.5 L) at 25-35° C. and maintained for 1 hour at same temperature. Filtered the reaction mass and washed with MTBE (3.0 L), suck dried to give the title compound.

Yield: 94.6

Purity: 98.69%

1. A one-pot process for the preparation of Tucatinib intermediate of Formula (VI) comprising: 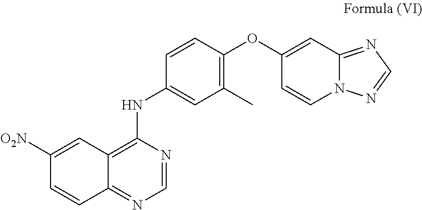 [SMILES: Cc1cc(Nc2ncnc3ccc([N+](=O)[O-])cc23)ccc1Oc1ccn2ncnc2c1]
(a) reacting a compound of Formula (III) with a suitable halogenating reagent to give a compound of Formula (IV); 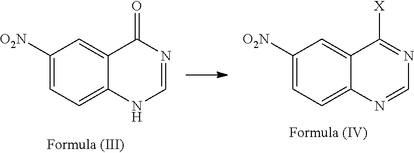
wherein X is Cl, Br, and I;
(b) coupling a compound of Formula (IV) with a compound of Formula (V) in acidic medium to produce a compound of Formula (VI) 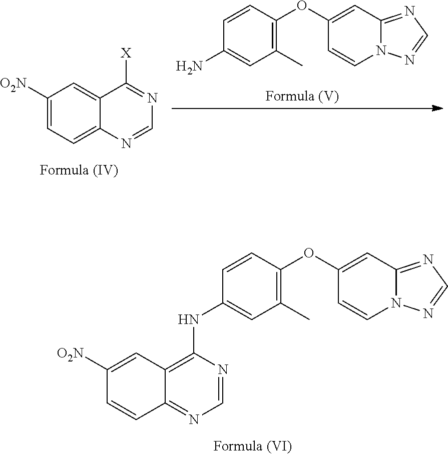
wherein X is defined above.

2. The process as claimed in claim 1, wherein suitable halogenating reagent is phosphorous oxychloride.

3. The process as claimed in claim 1, wherein X is Cl.

4. The process as claimed in claim 3, wherein the pH of the reaction mass is about 2-5.

5. The process as claimed in claim 1, wherein the compound of (VI) is converted to tucatinib.

6. A process for the preparation of tucatinib intermediate of Formula (VII) comprising transfer hydrogenation of compound of Formula (VI) to provide a compound of Formula (VII)

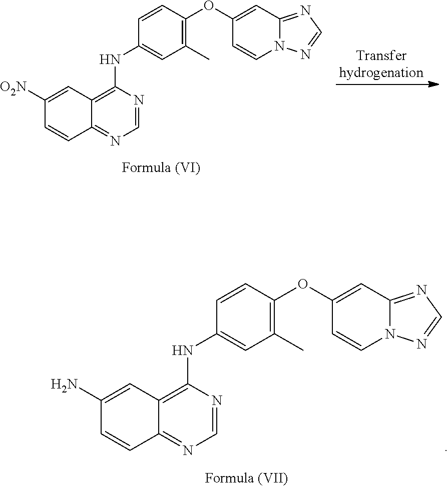 [SMILES: Cc1cc(Nc2ncnc3ccc([N+](=O)[O-])cc23)ccc1Oc1ccn2ncnc2c1 ; Cc1cc(Nc2ncnc3ccc(N)cc23)ccc1Oc1ccn2ncnc2c1]

7. The process as claimed in claim 6, wherein the transfer hydrogenation is carried out in presence of a metal catalyst and a suitable reagent.

8. The process as claimed in claim 7, wherein the metal catalyst is palladium.

9. The process as claimed in claim 7, wherein the suitable reagent is selected from a group of ammonium formate, triethyl silane, isopropyl alcohol, formic acid and hydrazine.

10. The process as claimed in claim 6, wherein the compound of (VII) is converted to tucatinib.

11. A process for the preparation of amorphous Tucatinib (I), comprising
a) coupling a compound of Formula (VII) with compound of Formula (VIII) or an acid-addition salt thereof in presence of an organic base to produce tucatinib Formula (I) or an acid-addition salt thereof; and 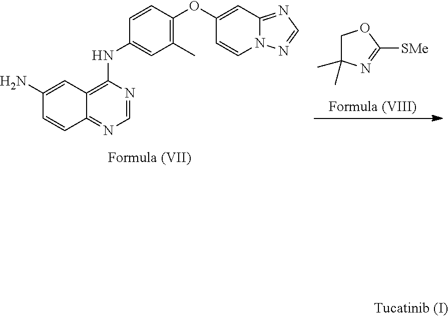
b) optionally, treating the acid-addition salt of compound of formula (I) with a base in a suitable solvent.

12. The process, as claimed in claim 11, wherein the organic base may be selected from a group of pyridine, 2-picoline, 3-picoline, 4-picoline, pyrrole, pyrrolidine, piperidine, and mixture thereof.

13. The process, as claimed in claim 11, wherein the step a) is carried out at about 30° C. to about 80° C.

14. The process, as claimed in claim 11, wherein the base used in step b) is ammonia.

15. A process for preparing amorphous tucatinib, comprising use of crystalline form of Tucatinib triflate of formula (IX), 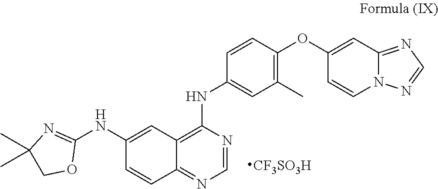
which is characterized by:
(i) Powder X-ray diffactogram having peaks at about 6.9, 8.5, 10.6, 13.9, 18.4, 21.3, 25.4±0.2 °2θ;
(ii) DSC thermogram as shown in FIG. 2.
